(19) United States
(12) Patent Application Publication
(10) Pub. No.: US 2026/0345247 A1
(43) Pub. Date: Nov. 5, 2026

(54) CONTROL DEVICE AND SYSTEM, AND SYSTEM HAVING A MEDICAL OPERATION INSTRUMENT, A DATA CAPTURE DEVICE AND A DATA PROCESSING APPARATUS

(71) Applicant: KARL STORZ SE & Co. KG, Tuttlingen (DE)

(72) Inventors: Florian HUBER, Tuttlingen (DE); Chunman FAN, Tuttlingen (DE); Mirko KUNZE, Tuttlingen (DE); Kirsten KLEIN, Tuttlingen (DE); Lena FELBER, Tuttlingen (DE); Thorsten AHRENS, Tuttlingen (DE); Hans-Georg MATHÉ, Tuttlingen (DE)

(73) Assignee: KARL STORZ SE & Co. KG, Tuttlingen (DE)

(21) Appl. No.: 18/997,330

(22) PCT Filed: Jul. 20, 2023
(86) PCT No.: PCT/EP2023/070175

§ 371 Date: Jan. 21, 2025

(30) Foreign Application Priority Data
Jul. 21, 2022 (DE) 10 2022 118 328.9
Jul. 21, 2022 (DE) 10 2022 118 330.0

(51) Int. Cl. A61B 34/20; A61B 1/04; A61B 1/313

(52) U.S. Cl. CPC A61B 34/20; A61B 1/04; A61B 1/3132; A61B 2034/2048; A61B 2034/2055

(57) ABSTRACT

The disclosure relates to a control device having a first optical sensor and a second sensor with an acceleration sensor and/or a gyroscope, wherein the optical sensor is designed to capture a first piece of information about a first translational and rotational component of a displacement of a first manually displaceable instrument relative to the optical sensor, the second sensor is designed to capture a second piece of information about a second component of the displacement of the first instrument, wherein the control device is designed to transmit the first and the second piece of information for positioning a second instrument such that the second instrument is displaced in correspondence with the first instrument. The disclosure also relates to a system having a control device, a first instrument, a second instrument and a motor-driven control unit for positioning the second instrument, and to a system having a medical operation instrument.

### CROSS-REFERENCE TO RELATED APPLICATIONS

This application is the U.S. national stage of PCT/EP2023/070175 filed on Jul. 20, 2023, which claims priority of German Patent Application No. DE 10 2022 118 328.9 filed on Jul. 21, 2022, and German Patent Application No. DE 10 2022 118 330.0 filed on Jul. 21, 2022, the contents of which are incorporated herein.

### TECHNICAL FIELD

The present disclosure relates to a control device and a system with a control device, a first instrument, a second instrument, and a motorized control unit for positioning the second instrument.

### BACKGROUND

In medical technology, there are multiple established methods for controlling a robot system. These include, for example, a joystick that is attached to a device with a screw. This type of control is mainly used to control a robotic arm, which in turn positions an endoscope. However, it can be inconvenient to reach the joystick, and controlling the endoscope feels unnatural.

Other robot systems use voice control as a control mechanism. This technology is very popular in certain markets (e.g., the USA), but, in other regions, this method is used less frequently. For example, voice control has a certain inaccuracy and delay. In addition, only one degree of freedom can be controlled at any given time. In addition, communication in the operating room must be subordinate to the voice-controlled system when the robotic arm is repositioned.

Another possibility is gesture tracking. Hands-free control of the robot arm is captured, for example, using a headband that has an infrared receiver. When the surgeon nods their head, the receiver captures the movement and commands the arm to reposition the endoscope accordingly.

It is also possible to use imaging algorithms that identify the tip of an instrument. The robot moves the instrument, in particular an endoscope, so that this tip is centered in the image. However, controlling the endoscope based upon video sequence analysis is challenging, because automatic object detection in the image is a very difficult issue, and deterministic behavior of the endoscope control cannot always be guaranteed in complex situations.

Finally, there is a new method called “eye tracking” that uses smart glasses (e.g., augmented reality glasses) to track the movements of the surgeon's eye, locate the intended visual target, and interpret the tracking information as commands to reposition the robotic arm or motorized endoscope holder.

### SUMMARY

It is an object of the present disclosure to provide an improved control device and an improved system that allows the user intuitive control.

The object is achieved according to one aspect by a control device having a first, optical sensor and a second sensor with an acceleration sensor and/or a gyroscope, wherein the optical sensor is designed to capture a first piece of information about a first translational and rotational component of a displacement of a first manually displaceable instrument relative to the optical sensor, and the second sensor is designed to capture a second piece of information about a second component of the displacement of the first instrument, wherein the second component is a roll, pitch, and yaw of the first instrument, wherein the control device is designed to transmit the first and the second pieces of information for positioning a second instrument such that the second instrument is displaced in correspondence with the first instrument.

Such a control device allows the user to move an instrument—the first instrument, which he is already operating—as desired. The reaction of the second instrument occurs immediately and in accordance with the movement of the first instrument, so that the user achieves an intuitive and direct movement of the second instrument by moving the first instrument. In particular, the first and second pieces of information are sent directly or indirectly to a motorized control unit, which is designed to position the second instrument.

In an advantageous embodiment, the control device has a continuous passage which is designed to receive a shaft of the first instrument.

This design enables a defined interaction between the control device and the first instrument.

In a further advantageous embodiment, the optical sensor is arranged such that light reflected from the first instrument reaches the optical sensor.

This design makes it possible to reliably capture the translational and rotational components of the displacement of the first instrument. In particular, the passage is in the field of view of the optical sensor.

In a further advantageous embodiment, the control device has a light source which is designed to emit light for illuminating a shaft of the first instrument.

This design makes it possible to reliably capture the translational and rotational components of the displacement of the first instrument. The light is emitted in particular in the direction of the passage and is at least partially reflected there by a shaft of the first instrument, in the direction of the optical sensor.

In a further advantageous embodiment, the light is emitted with a structured pattern.

This design makes it possible to reliably capture the translational and rotational components of the displacement of the first instrument.

In a further advantageous embodiment, the optical sensor is arranged in a hermetically sealed housing with a light-permeable pane.

This design allows the optical sensor to be protected from external influences, but still be able to capture or observe the translational and rotational components of the displacement of the first instrument. This pane is preferably either arranged at a certain angle to the surface of the optical sensor or is provided with an anti-reflective coating of 800 to 900 nm to prevent reflections from the pane from interfering with the optical sensor.

In a further advantageous embodiment, the control device has an actuating device, wherein the control device is designed, in an activated state of the actuating device, to effect a displacement of the second instrument when the first instrument is displaced, and, in a deactivated state, to effect no displacement of the second instrument when the first instrument is displaced.

This configuration allows the user to easily switch to a first operating mode to move the second instrument using the first instrument, or to switch to a second operating mode to work with the first instrument without moving the second instrument. For example, the user can, in the first operating mode, first position an endoscope with a camera using the first instrument, then, in the second operating mode, work with the first instrument, then again, in the first operating mode, reposition the endoscope, and so on.

In a further advantageous embodiment, the control device has a configuration device which is designed to apply a factor to the first and/or the second piece of information, so that a displacement of the first instrument with respect to one or more degrees of freedom takes place in a displacement of the second instrument with respect to this or these degrees of freedom, to which a factor is applied.

This design makes it possible to configure a translation of the first displacement into the second displacement. For example, if insertion of the first instrument is intended to lead only to a smaller insertion of the second instrument, a positive factor less than 1 can be set for this degree of freedom. If a displacement of the first instrument is intended to generally lead to smaller displacements of the second instrument, a factor smaller than 1 is chosen for all degrees of freedom. If a displacement of the first instrument is intended to generally lead to larger displacements of the second instrument, a factor greater than 1 is chosen for all degrees of freedom. Using a negative factor, a mirror-image translation can be selected for one or more degrees of freedom if desired by the user.

In a further advantageous embodiment, the control device further has an instrument trocar to which the control device is fixed.

This configuration enables a defined interaction between the control device and the first instrument.

In a further advantageous embodiment, a first reference coordinate system of the first instrument is formed from three axes that are perpendicular to one another pairwise, wherein a z-axis of the three axes is directed from a distal end of the first instrument towards a proximal end of the first instrument, an x-axis of the three axes follows the gravitational field of the earth, and a y-axis of the three axes is perpendicular to the x-axis and the z-axis.

This design enables a practical and computationally easy-to-realize definition of the first reference coordinate system of the first instrument.

In a further advantageous embodiment, a second reference coordinate system of the second instrument is formed from three axes perpendicular to each other pairwise, wherein a z-axis of the three axes is directed from a distal end of the second instrument towards a proximal end of the second instrument, an x-axis of the three axes follows the gravitational field of the earth, and a y-axis of the three axes is perpendicular to the x-axis and the z-axis.

This design enables a practical and computationally easy-to-realize definition of the second reference coordinate system of the second instrument.

In the following, designs of a preferred concrete embodiment are described, wherein individual elements of this embodiment can also be selectively combined with the control device described in general above.

In this specific embodiment, a sensor unit is positioned near an instrument trocar and kinetically coupled thereto, wherein the instrument trocar is designed to receive the first instrument. The sensor unit comprises an optical sensor, in particular a tracking sensor, and a 3-axis accelerometer and/or a 3-axis gyroscope. The optical tracking sensor is arranged to provide a direct optical access path to the instrument shaft when the first instrument is inserted into the instrument trocar, while the sensor is enclosed in a hermetic housing.

The accelerometer and/or gyroscope capture the absolute roll and pitch movement as well as the relative yaw movement of the sensor unit, and thus of the instrument trocar. The optical tracking sensor captures the translation and rotation of the first instrument. This sensor has a laser-based light source that projects a pattern onto the surface of the instrument shaft of the first instrument. The hermetic housing of the optical tracking sensor has a glass window that allows optical access to the instrument shaft. This window is preferably either arranged at a certain angle to the surface of the sensor or provided with an anti-reflective coating of 800 to 900 nm to prevent reflections from the glass from disturbing the sensor.

In addition, the control device or system has an activation method that signals when the user starts the endoscope control mode. This activation method can, for example, be a foot switch, a button on the instruments, or a voice control. In addition, there is a connection between the second instrument—here, a robotic arm with an endoscope—and the first instrument, which defines exactly how the endoscope and the camera are positioned relative to the endoscope positioner—here, the robotic arm.

This enables a direct and intuitive assignment of particular degrees of freedom. The inclination of the first instrument can easily be translated into the inclination of the second instrument or of the endoscope, and the translation of the instrument shaft can be mapped to a translation of the second instrument or of the endoscope.

However, this sensor arrangement has no information about the absolute orientation of the first instrument in the horizontal plane, or how the first instrument is positioned in relation to the second instrument or the endoscope or how the user or surgeon is positioned in relation to the first instrument or the second instrument—here, the endoscope. Therefore, a direct association between the movement of the first instrument and that of the second instrument is not possible. Therefore, the aforementioned first piece of information and second piece of information are captured and are used as the basis for calculating which movement of the first instrument leads to an endoscope movement to the right or to the left.

The proposed mapping method, which can be applied to all embodiments described in this disclosure, requires neither user calibration nor additional sensors. Instead, the gravity vector and the instrument axis are used to calculate the left-right direction according to the right-hand rule. The axis of gravity can be represented by the thumb and the direction of insertion of the instrument by the index finger. Then, the direction in which the middle finger points is considered to be to the right. When the user moves the instrument tip to the right, the camera moves to the right side of the image. Accordingly, the direction of rotation can also be determined using the right-hand rule. The thumb can point in the direction of insertion. Then, the bent fingers point in the direction that causes the image on the screen to rotate clockwise.

Such considerations can be carried out in both a Cartesian and a spherical coordinate system. In the latter case, the sensor unit forms a spherical coordinate system with the sensor unit at its center (the position or horizontal orientation of the sensor unit are irrelevant). The tip of the first instrument can be considered a point in the coordinate system, and the instrument shaft its vector. Moving the tip in the positive direction of the azimuth unit vector causes the image to move to the left, while moving the tip in the direction of the positive polar angle unit vector causes the image to move downwards. Moving the tip in the positive direction of the radius unit vector causes a zoom movement.

This mapping method is not chosen arbitrarily; rather, it has been identified as preferred because it is based upon the surgeon's daily experience. If the first instrument were actually an endoscope with a camera, it would move the image on the screen in the same way that this mapping method does when the surgeon uses the first instrument to control the image rather than the endoscope directly. This will simplify the learning process for trained surgeons. In addition, the mapping method is based upon the instrument itself. Every person in the operating room can understand which instrument movement on the first instrument causes which endoscope movement, i.e., movement of the second instrument. This allows anyone in the operating room to easily control the system, regardless of their positioning in relation to the monitor, the first instrument and second instrument, or of the endoscope or camera.

In this context, reference is made to the explanations at https://www.scratchapixel.com/lessons/mathematics-physics-for-computer-graphics/lookat-function.

According to a further aspect, the object is achieved by a system with a previously described control device, a first instrument, a second instrument, and a motorized control unit for positioning the second instrument.

In an advantageous embodiment, the first instrument is designed to be guided in an instrument trocar.

In a further advantageous embodiment, the second instrument is an endoscope, wherein the endoscope in particular has a camera.

The present disclosure also relates to a system having a medical operation instrument, a data capture device, and a data processing apparatus.

The capture of data during an operation has increased in importance. Documentation of the operation has long been a recognized requirement. However, it is still a challenge today to document an operation in such a way that details of the operation can also be understood even after the operation has been completed.

Over time, however, the information technology support of an operation has also become more important. For example, a specific phase of an operation can be captured by activating a specific instrument. This information can then be processed immediately, i.e., during the operation—for example, to make certain settings on one or more instruments or devices.

In practice, however, it turns out again and again that information technology support does not always provide a complete overall picture of an operation. It is therefore an object of the present disclosure to provide an improved system which at least supports the execution or documentation of the operation and optionally opens up further possibilities for obtaining information.

As a solution, a system is proposed with a medical operation instrument that is designed to be manually guided by the surgeon at least temporarily during an operation, a data capture device, and a data processing apparatus, wherein the data capture device has a pose detector that is designed to repeatedly capture a piece of pose information of the operation instrument, wherein the pose information has at least one piece of position or orientation information, and to send it to the data processing apparatus, and wherein the data processing apparatus is designed to process the pose information and to support the execution or documentation of the operation.

A special feature of this approach is that the operation instrument is no longer viewed only as an actuator, whether manually or robotically guided, but is used as an input medium to collect further important information. The pose detector can be designed as a separate device that can be releasably fixed to the operation instrument.

The pose detector can also be an integrated part of a motorized device. This device may already have one or more detectors that determine and, if necessary, monitor the pose of the device and thus of the operation instrument in space.

However, the pose detector can also be inherently formed by the motorized device. It is a fact that such a device, in particular a motorized robot arm, can assume specific positions in space through specific positioning of at least one motor. If the position of the device is then manually changed, the setting position of the at least one motor also changes, which in turn allows conclusions to be drawn about the current pose of the operation instrument.

In this case, the known causality—in which a desired, specific position is specified, the setting position of the at least one motor is controlled, and thus the specific position is set in the space of the motor-operated device—is reversed. The position in space now becomes a selectable parameter that specifies the setting position of the at least one motor, from which the position in space can be determined.

One aspect of the present disclosure addresses a topic that the inventors have recognized in connection with the present disclosure. This issue will be referred to below as a data gap, especially as a data gap for manual operation steps.

Surgical robots are used only for certain parts of the operation, depending upon the procedure, the methodology, and the surgeon. While the beginning and end of the operation are always carried out on the operating table, preparatory steps such as preparation before resection or intermediate steps in the case of complex procedures can also be carried out manually on the operating table. During this manual phase, significantly less intervention-related data can be collected than when using the robot.

By way of example, this concerns one or more elements from the group comprising the type of instrument used, the instrument position, the change in the instrument position, the instrument condition, the instrument use, the service life of an instrument, the camera position, the change in the camera position, the relative position between camera(s) and instruments, the relative movement between camera(s) and instrument(s), the relative acceleration between camera(s) and instrument(s), collisions between camera(s) and/or instrument(s).

However, intervention-related data collected at the robot can be used to generate medically and economically relevant added value. The disclosure can advantageously reduce or close these data gaps during the manual phases at the operating table.

It is therefore proposed to carry out continuous data collection during a surgical procedure that is partially performed in telemanipulated fashion or with a telemanipulator. For this purpose, a sensor unit is attached to the trocar and kinetically coupled to capture the instrument position in the operating room. This can be done with any number of trocars.

By combining information on the instrument type, including, for example, jaw geometry and/or kinematics, etc., and the instrument position, a digital image of reality can be created and captured and stored for further processing, as is the case when surgical robots are used.

Possible applications include the following aspects: automated documentation, recognition of phases during the operation, detection of emergency situations, e.g., due to an instrument movement, monitoring of the registered pivot points, evaluations for training purposes, integration of expert systems, and retrieval or control of exact positions, e.g., when changing from robotic guidance to manual guidance or vice versa.

In addition, a security feature can be implemented, which allows, for example, the definition of restricted areas. A partially robotically manipulated instrument cannot be moved into these restricted areas, even with manual operation, or can be moved into them only against increased resistance. This is achieved by appropriate control of a motorized robot arm, which is part of a surgical robot or telemanipulator.

A further aspect of the present disclosure is devoted to another topic which the inventors have recognized in connection with the present disclosure. This topic will be referred to below as the input unit, in particular an input unit for controlling an endoscope on the operating table.

With surgical robots, the focus is on the work of the main surgeon at the console. Nevertheless, it is necessary to carry out work steps manually at the operating table. This is done either by the main surgeon before working at the console or by the assistant during the robotic procedure. The robot's supporting functions are not available in these manual phases. This includes, for example, the tracking of the camera.

Therefore, for the manual phases at the operating table and for parallel work by the main surgeon at the console and the assistant at the operating table, a table-based input system for camera control shall temporarily replace the input at the console, so that the full range of functions of the robot for camera control, including automatic tracking, is still available at the operating table.

A device and a method are therefore proposed in which, via the console of the telemanipulator and also the evaluation of the input at the laparoscopic instrument, a parallel control of a robotically guided endoscope by the console of the telemanipulator as well as the evaluation of the input at the laparoscopic instrument, including, for example, control of the endoscope on the robot by a manual instrument.

A further aspect of the present disclosure is devoted to another topic which the inventors have recognized in connection with the present disclosure. This topic will be referred to below as a safety system, in particular as a redundant safety system.

A requirement for the approval of a medical product is an adequate safety design. For critical functions, this is usually based upon a redundant design. Arm and instrument positions are currently captured and calculated mainly via the sensors in the robot arm joints.

The integration of a sensor system, in particular a sensor system arranged near a trocar or near a puncture site, enables an independent additional measurement modality, so that the sensors can be used to monitor a safety system of a surgical robot. For example, shear forces in the registered trocar point can be detected that can occur when the position or registration of the pivot point or the puncture site is changed. Such a situation can occur, for example, due to a movement of the patient.

It is therefore proposed to capture an instrument position by means of a sensor system, in particular a sensor system arranged near a trocar or near a puncture site, in order to be able to compare the actual and target values of the kinematics of the surgical robot, in particular of at least one arm joint position of the surgical robot.

With regard to the aspects mentioned above, it should be noted that this further data capturing does not take place in the instrument itself. Here, the capturing is essentially limited to the tip of the instrument, so that, for example, an absolute position cannot be easily captured; only direct quantities such as forces, temperature, etc., can be captured. Instead, data collection is carried out here, in particular at a pivot point or using a passive mount.

Preferably, one or more of the following values are determined: absolute position, relative position in combination with the current image from the camera, motion curves, kinematics, dynamics, and forces. The latter can be calculated in particular from a combination of image and absolute position, e.g., how much an instrument bends under the weight of the liver.

In one embodiment, the data processing apparatus is designed to determine movement patterns of the surgeon.

The surgeon, i.e., the person operating the instrument the pose of which is being monitored, is observed or tracked as to how he works with the operation instrument. In particular, conclusions can be drawn about the surgeon's current condition, the so-called “surgeon mood.” The operation instrument can be freely manually guided or coupled to a motorized device in which the surgeon can guide the operation instrument substantially without resistance.

In a further embodiment, the data processing apparatus is designed to determine a tremor in the guiding of the instrument.

Intentional rhythmic movements can be identified here, e.g., based upon their frequency or deflection, and can be distinguished from a possible tremor of the surgeon. The identification of a tremor may indicate, for example, a strenuous holding of the instrument or tension on the part of the surgeon, and can be signaled to the surgeon or another person.

In a further embodiment, the data processing apparatus is designed to capture signs of stress and/or feeling rushed in the surgeon.

Both absolute limit values, e.g., too high a speed of a movement, excessive deflection, entering restricted areas or spaces, etc., as well as relative changes in an empirically determined movement pattern of the surgeon or group of surgeons can be determined. For example, increased speed, excessive movements, or deviation from usual routes or spaces, etc., can be signs of stress. This can be signaled to the surgeon or another person.

In a further embodiment, the data processing apparatus is designed to capture signs of fatigue in the surgeon.

For example, insufficient movement dynamics or reaction times that are too long may indicate that the surgeon has become tired. Here, too, absolute limit values can be determined, e.g., too low a speed of a movement, staying in one position for too long with no activity, etc., as well as relative changes in an empirically determined movement pattern of the surgeon or a group of surgeons.

In a further embodiment, the data processing apparatus is designed to determine a three-dimensional camera position even if only a two-dimensional image is available.

The additional information captured means that measurements, such as distance, path, or area measurements, can be carried out even in a two-dimensional image. In addition, a warning can be given that critical structures may be affected or will be affected if the movement continues. In this way, possible collisions or ruptures can be warned against. As already explained, also by controlling a robot arm accordingly, resistance to a potentially dangerous movement can be built up, or the movement can be blocked.

In a further embodiment, the data processing apparatus is designed to control the image tracking of a camera.

This can confer advantages, in particular in terms of data quality, but can also be a security feature. Image tracking can be carried out in particular by a combination of image recognition and the pose information, in particular position data, so that the surgeon has the relevant regions in view. It can also be ensured that all instruments are displayed in the visible image portion. Alternatively, the position of instruments that are not visible can be shown in an extended display.

In a further embodiment, the data processing apparatus is designed to determine a usage model.

The determined usage model or operator model can include a technical load assessment of the instruments, which can be used to make predictions about the service life or remaining service life of one or more instruments. Typical movement data of the surgeon can also be determined, and/or a cleaning intensity can be determined.

In a further embodiment, the data processing apparatus is designed to create a learning curve analysis from movement data.

The repeated capturing of the pieces of pose information in conjunction with time capturing is used to capture times, paths, and possibly contacts with and/or proximity to critical structures. Based upon this data, it can subsequently be determined where the surgeon can improve. In addition, any unsuccessful steps of the operation can be reproduced in a simulation, in particular by virtually recreating the situation, and improved in a training context.

In a further embodiment, the data processing apparatus is designed to support a non-surgeon.

The term non-surgeon should be understood as referring to a person who does not perform or significantly shape the medical procedure. This may in particular be an assisting person, e.g., an assistant at the table. The data processing apparatus can thus control an image processing in which the position of the assistant's instrument is superimposed in a display to the assistant, in particular if the instrument is not visible in the image display currently desired by the surgeon. It can also represent a hidden direction that is not visible in the image. Finally, the assistant can be supported in capturing or picking up an instrument with the camera at the trocar exit, and accompanying the return of the instrument to the target structure.

In a further embodiment, the data processing apparatus is designed to automatically create reports and/or to perform a workflow analysis.

The movement data are used in the automatic division of the procedure into different phases. On the one hand, this can be done after completion of an operation (post-op). In particular, an automated operation report can be created, and/or, by stitching multiple images together, it can be documented that the entire site has been inspected. On the other hand, workflow optimization can also be carried out during the operation (intra-op), such as determining the time of appointment for the next patient. Surgical delays can be determined in comparison to the hospital's average values, and, based upon such a determination, a possible automated adjustment of the surgical plan can be made.

In a further embodiment, the data processing apparatus is designed to control a desired pose of the operation instrument.

This can make it easier to change or make adjustments on the console and/or at the operating table. In particular, an instrument position can be controlled again when changing from manual to robotic guidance. In a similar way, a camera position can again be assumed.

It is understood that the features mentioned above and those to be explained below can be used not only in the combination specified in each case, but also in other combinations or on their own, without departing from the scope of the present disclosure.

In particular, it is to be noted that the aspects of the various systems described can be combined both with the control device described and with one or more of the other systems described.

### BRIEF DESCRIPTION OF THE DRAWINGS

The drawings, the description, and the claims contain numerous features in combination. It is understood that the features mentioned above and those to be explained below can be used not only in the combination specified in each case, but also in other combinations or on their own, without departing from the scope of the present disclosure. An exemplary embodiment of the disclosure is shown in the drawing and is explained in more detail in the following description. Shown is:

FIG. 1 an embodiment of a control device as part of an embodiment of a system.

### DETAILED DESCRIPTION OF PREFERRED EMBODIMENTS

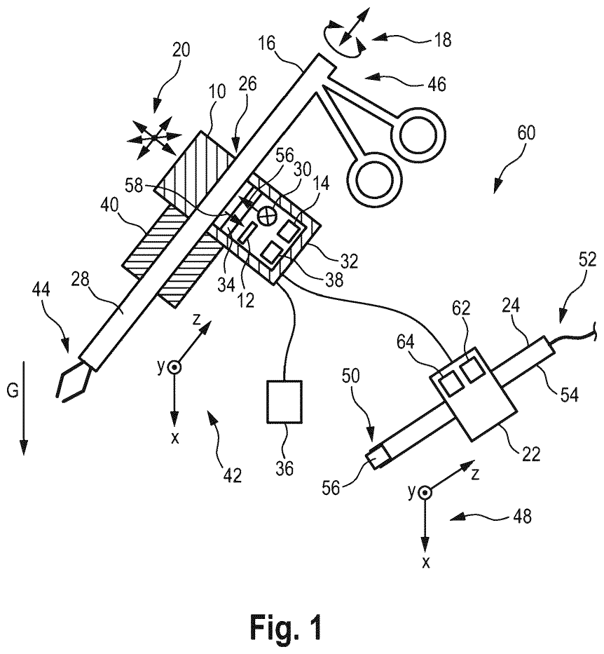

FIG. 1 shows an embodiment of a control device 10, which is part of a system 60, which further has a first instrument 16, a second instrument 24, and a motorized control unit 22 for positioning the second instrument 24. The first instrument 16 is designed to be guided in an instrument trocar 40. The second instrument 24 here is an endoscope 54, wherein the endoscope 54 in particular has a camera 56. The control device 10 is detachably fixed to the instrument trocar 40.

The control device 10 has a first, optical sensor 12 and a second sensor 14 with an acceleration sensor and/or a gyroscope, wherein the optical sensor 12 is designed to capture a first piece of information about a first translational and rotational component 18 of a displacement of a first, manually displaceable instrument 16 relative to the optical sensor 12.

The second sensor 14 is configured to capture a second piece of information about a second component 20 of the displacement of the first instrument 16, wherein the second component 20 is a roll, pitch, and yaw of the first instrument 16.

The control device 10 is designed to transmit the first and second pieces of information to the motorized control unit 22 for positioning a second instrument 24 so that the second instrument 24 is displaced in correspondence with the first instrument 16. The second instrument 24 is releasably fixed to the motorized control unit 22.

The control device 10 has a continuous passage 26 which is designed to receive a shaft 28 of the first instrument 16, in particular in a positively fitting manner with respect to the circumference of the shaft 28. The optical sensor 12 is arranged such that light 58 reflected from the first instrument 16 reaches the optical sensor 12. The optical sensor 12 is arranged in a hermetically sealed housing 32 with a light-permeable pane 34. The light 56 comes in particular from a light source 30 which is designed to emit light 56 for illuminating a shaft 28 of the first instrument 16. The light 56 is emitted in particular with a structured pattern.

The control device 10 has an actuating device 36, wherein the control device 10 is designed, in an activated state of the actuating device 36, to effect a displacement of the second instrument 24 when the first instrument 16 is displaced, and, in a deactivated state, to effect no displacement of the second instrument 24 when the first instrument 16 is displaced.

The control device 10 also has a configuration device 38 which is designed to apply a factor to the first and/or the second pieces of information, so that a displacement of the first instrument 16 with respect to one or more degrees of freedom leads to a displacement of the second instrument 24 with respect to this or these degrees of freedom, to which a factor is applied.

A first reference coordinate system 42 of the first instrument 16 is formed from three axes perpendicular to each other pairwise, wherein a z-axis of the three axes is directed from a distal end 44 of the first instrument 16 towards a proximal end 46 of the first instrument 16, an x-axis of the three axes follows the gravitational field G of the earth, and a y-axis of the three axes is perpendicular to the x-axis and the z-axis.

A second reference coordinate system 48 of the second instrument is formed from three axes perpendicular to each other pairwise, wherein a z-axis of the three axes is directed from a distal end 50 of the second instrument 24 towards a proximal end 52 of the second instrument 24, an x-axis of the three axes follows the gravitational field G of the earth, and a y-axis of the three axes is perpendicular to the x-axis and the z-axis.

The motorized control unit 22 has a processing unit 62—here, a microprocessor—and a memory 64. The memory 64 has instructions with which, when these instructions are executed by the processing unit 62, control commands for the motorized control unit 22 are determined from the first and second pieces of information.

A preferred implementation in terms of programming of the controlling by the control device is described below. It is assumed here that the first instrument—here, in the function of a joystick—controls a camera of an endoscope, which represents the second instrument. When this type of control is active, the system is in a camera control mode (CCM). Variables assigned to the camera have the identifier “cam.” Variables assigned to the joystick have the identifier “joy.”

When the camera control mode is switched on, the following initial values are saved in variables. This is necessary because all control commands are calculated from the difference between these initial values and the new measured values; see Table 1:

TABLE 1

| | |
|---|---|
| phaseStartJoyOri = joyOri | joyOri = current orientation of the |
| | instrument |
| phaseStartJoyRollMouse = | joyRoll = current roll value |
| joyRoll | |
| phaseStartJoyInsertion = | joyInsertion = how far the instrument was |
| joyInsertion | inserted |
| phaseStartCamOri = camOri | camOri = orientation camera/endoscope |
| phaseStartCamInsertion = | camInsertion = how far the |
| camInsertion | camera/endoscope was inserted |
| rollSum = 0 | rollSum = additional variable for calculat- |
| | ing the incremental change of rotation |

If the camera control mode has been activated and initialized as previously explained, first, the first reference coordinate system of the first instrument, i.e., the joystick, is determined.

As already mentioned, the z-axis is defined by the instrument and points from the tip of the instrument to the handle. In principle, this axis can be mathematically assigned a negative sign, which results in a reversal of the direction. However, the orientation suggested here is standard in camera technology; see in particular the so-called LookAt function.

As already described, two further axes are defined. To define these clearly, the gravitational axis is used. This is always known due to the inertial sensors and can therefore be used. This axis, which follows the gravitational field, will be referred to here as the x-axis for the sake of clarity. The y-axis is ultimately the cross-product of the x-axis and the z-axis. This is shown in Table 2 below:

TABLE 2

| | |
|---|---|
| RefZ = phaseStartJoyOri | Definition: Unit vector from the instrument |
| | tip along the shaft to the handle is the z-axis |
| RefX = Norm([0, 0, 1] × | [0, 0, 1] is the vector of the gravitational axis |
| RefZ) | G or the x-axis. |
| RefY = RefZ × RefX | Cross-product gives y-axis (formula: |
| | RefY = RefZ × RefX) |
| joyRef = np.eye(4) | Output of the coordinate system in a matrix |
| joyRef [0:3, 0] = RefX | |
| joyRef [0:3, 1] = RefY | |
| joyRef [0:3, 2] = RefZ | |
| return joyRef | |

The calculation of the second reference coordinate system of the second instrument—here, the camera or the endoscope—is carried out in the same way as for the first instrument, wherein the data of the endoscope are now used here; see Table 3:

TABLE 3

| | |
|---|---|
| RefZ = phaseStartCamOri | Definition: Unit vector from endoscope tip |
| | along the shaft to the handle |
| RefX = Norm([0, 0, 1] × | [0, 0, 1] is the vector of the gravitational |
| RefZ) | axis. |
| RefY = RefZ × RefX | Cross-product gives y-axis |
| CamRef = np.eye(4) | Output of the coordinate system in a matrix |
| CamRef [0:3, 0] = RefX | |
| CamRef [0:3, 1] = RefY | |
| CamRef [0:3, 2] = RefZ | |
| return CamRef | |

Now that the first and second reference coordinate systems have been determined, the following steps are carried out continuously (see Table 4), from which important parameters for controlling the second instrument are then calculated-preferably deflection, deflection direction, roll value, and insertion depth:

TABLE 4

| | |
|---|---|
| invJoyRef = inv(joyRef) | The inverse of the first reference |
| | coordinate system is calculated. |
| joyRelative_0 = invJoyRef × | Calculates the orientation of the first |
| phaseStartJoyOri | instrument in the first reference coordinate |
| | system at the time of starting the CCM |
| joyRelative = invJoyRef × | Current orientation of the first instrument |
| joyOri | in the first reference coordinate system |

The drawings, the description, and the claims contain numerous features in combination. It goes without saying that the above-mentioned features can be used not only in the combination specified in each case, but also in other combinations or on their own, without departing from the scope of the present disclosure. The disclosure relates to a control device (10) with a first, optical sensor (12) and a second sensor (14) with an acceleration sensor, and/or a gyroscope, wherein the optical sensor (12) is designed to capture a first piece of information about a first translational and rotational component (18) of a displacement of a first manually displaceable instrument (16) relative to the optical sensor (12), and the second sensor (14) is designed to capture a second piece of information about a second component (20) of the displacement of the first instrument (16), wherein the control device (10) is designed to transmit the first and the second pieces of information for positioning a second instrument (24) so that the second instrument (24) is displaced in correspondence with the first instrument (16). Furthermore, a system (60) with a control device (10), a first instrument (16), a second instrument (24), and a motorized control unit (22) for positioning the second instrument (24), as well as a system with a medical operation instrument, are disclosed.

1. A control device with a first optical sensor and a second sensor with an acceleration sensor and/or a gyroscope, wherein the optical sensor is designed to capture a first piece of information about a first translational and rotational component of a displacement of a first manually displaceable instrument relative to the optical sensor, and the second sensor is designed to capture a second piece of information about a second component of the displacement of the first instrument, wherein the second component is a roll, pitch, and yaw of the first instrument, wherein the control device is designed to transmit the first and the second pieces of information for positioning a second instrument so that the second instrument is displaced in correspondence with the first instrument.

2. The control device according to claim 1, wherein the control device has a continuous passage designed to receive a shaft of the first instrument.

3. The control device according to claim 1, wherein the optical sensor is arranged such that light reflected from the first instrument reaches the optical sensor.

4. The control device according to claim 1, wherein the control device has a light source which is designed to emit light for illuminating a shaft of the first instrument.

5. The control device according to claim 4, wherein the light is emitted with a structured pattern.

6. The control device according to claim 1, wherein the optical sensor is arranged in a hermetically sealed housing with a light-permeable pane.

7. The control device according to claim 1, wherein the control device has an actuating device, wherein the control device is designed, in an activated state of the actuating device, to effect a displacement of the second instrument when the first instrument is displaced, and, in a deactivated state, to effect no displacement of the second instrument when the first instrument is displaced.

8. The control device according to claim 1, wherein the control device has a configuration device which is designed to apply a factor to the first and/or the second piece of information, so that a displacement of the first instrument with respect to one or more degrees of freedom leads to a displacement of the second instrument with respect to this or these degrees of freedom, to which a factor is applied.

9. The control device according to claim 1, further having an instrument trocar to which the control device is fixed.

10. The control device according to claim 1, wherein a first reference coordinate system of the first instrument is formed from three axes perpendicular to one another pairwise, wherein a z-axis of the three axes is directed from a distal end of the first instrument towards a proximal end of the first instrument, an x-axis of the three axes follows the gravitational field of the earth, and a y-axis of the three axes is perpendicular to the x-axis and the z-axis.

11. The control device according to claim 1, wherein a second reference coordinate system of the second instrument is formed from three axes perpendicular to one another pairwise, wherein a z-axis of the three axes is directed from a distal end of the second instrument towards a proximal end of the second instrument, an x-axis of the three axes follows the gravitational field of the earth, and a y-axis of the three axes is perpendicular to the x-axis and the z-axis.

12. A system with a control device according to claim 1, a first instrument, a second instrument, and a motorized control unit for positioning the second instrument.

13. The system according to claim 12, wherein the first instrument is designed to be guided in an instrument trocar.

14. The system according to claim 12, wherein the second instrument is an endoscope, wherein the endoscope in particular has a camera.

15. A system with a medical operation instrument which is designed to be manually guided by the surgeon at least temporarily during an operation, a data capture device, and a data processing apparatus, wherein the data capture device has a pose detector which is designed to repeatedly capture a piece of pose information of the operation instrument, wherein the pose information has at least one piece of position or orientation information, and to transmit it to the data processing apparatus, and wherein the data processing apparatus is designed to process the pose information and to support the execution or documentation of the operation.
